(19) United States
(12) Patent Application Publication
(10) Pub. No.: US 2026/0193182 A1
(43) Pub. Date: Jul. 9, 2026

(54) Preparation Method for Atorvastatin Calcium

(71) Applicant: CHONGQING PUYOU BIOPHARMA CO., LTD., Chongqing (CN)

(72) Inventor: Tao JIANG, Chongqing (CN)

(73) Assignee: CHONGQING PUYOU BIOPHARMA CO., LTD., Chongqing (CN)

(21) Appl. No.: 18/863,750

(22) PCT Filed: Nov. 30, 2022
(86) PCT No.: PCT/CN2022/135696

§ 371 Date: Nov. 7, 2024

(30) Foreign Application Priority Data
Aug. 17, 2022 (CN) 202210984987.X

(51) Int. Cl. C07D 207/34

(52) U.S. Cl. CPC C07D 207/34

(57) ABSTRACT

The present disclosure relates to the technical field of organic synthesis, in particular to a preparation method for atorvastatin calcium. The method including: using 2-((2R,4R)-4-hydroxy-6-oxotetrahydro-2H-pyran-2-yl)acetonitrile as a raw material, reacting same with 2,2-dimethoxypropane to add a protecting group, then performing hydrogenation reduction, docking same to a main nucleus, and finally hydrolyzing same into a salt to prepare the atorvastatin calcium. Compared with existing methods, the preparation method of the present disclosure has mild reaction conditions, convenient operations, high yield and purity, low costs, and is suitable for mass industrial production.

### CROSS-REFERENCE TO RELATED APPLICATIONS

This application is based on Chinese Patent Application No. 202210984987.X, filed Aug. 17, 2022, which claims the benefit of priority to the Chinese Patent Application, which is incorporated by reference in its entirety herein.

### TECHNICAL FIELD

The present disclosure relates to the field of pharmaceutical intermediates, in particular to a preparation method for atorvastatin calcium.

### BACKGROUND

Atorvastatin calcium, with chemical name [R—(R′,R′)]-2-(4-fluorophenyl)-β,α-dihydroxy-5-(1-methylethyl)-3-phenyl-4-[(phenylamino)carbonyl]-1-hydro-pyrrole-1-calcium enanthate (2:1) trihydrate and Chemical Abstracts Service (CAS) Number 134523-03-8, is a selective and competitive inhibitor of HMG-CoA reductase, which may treat the elevation of total cholesterol, low-density lipoprotein cholesterol, apolipoprotein B, and triglyceride.

A route used by U.S. Pat. No. 5,155,251A and WO2007096751A1 is as follows.

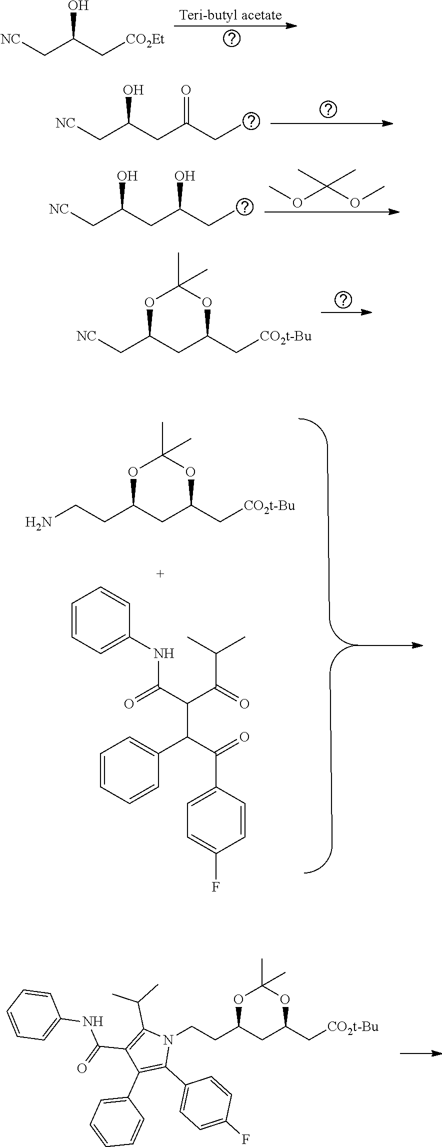

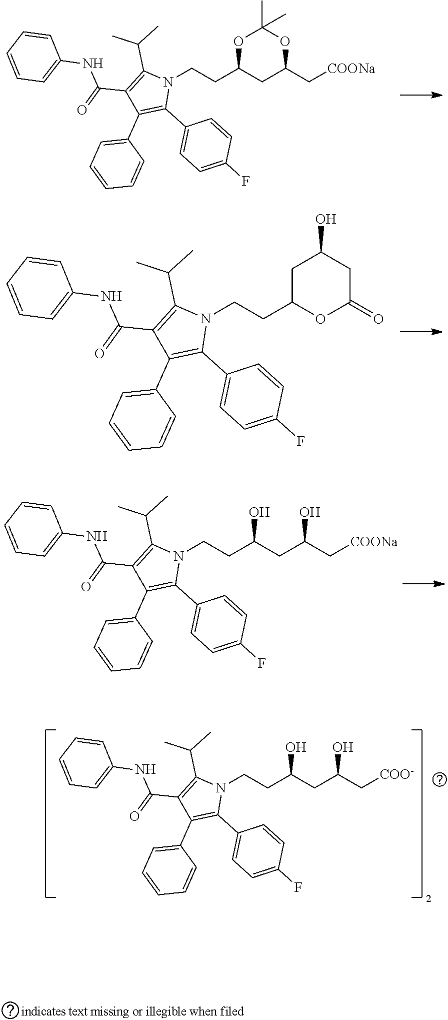

The reagents used in the route are relatively expensive, the reaction conditions are harsh, and the amount of waste generated is relatively large, resulting high costs and making it is unsuitable for industrial production.

### SUMMARY

In order to solve problems in the above technologies, the present disclosure provides a preparation method for atorvastatin calcium, which is simple in overall synthesis route, convenient in operation, high in yield and purity, and suitable for mass industrial production.

In order to achieve the above purposes, the present disclosure provides the following technical solution.

A preparation method for atorvastatin calcium is provided, and the atorvastatin calcium is as follows:

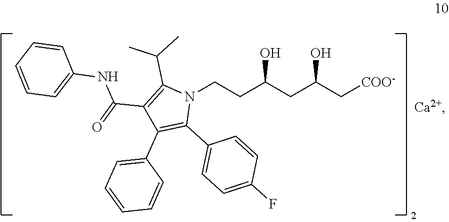

the preparation method includes the following steps:
(1) a compound 1 is ring-cleaved by an ammonia compound to obtain a compound 2;
(2) the compound 2 is reacted with 2,2-dimethoxypropane in a catalytic reaction with an acid to obtain a compound 3;
(3) the compound 3 is catalytic-hydrogenated by Raney nickel under a pressure to obtain a compound 4;
(4) the compound 4 is condensed with a compound 5 to obtain a compound 6;
(5) the compound 6 is hydrolyzed to obtain a compound 7;
(6) the compound 7 is subjected to acid deprotection and acid catalytic dehydration to obtain a compound 8, while the compound 7 is subjected to the acid deprotection to obtain a compound 9;
(7) the compound 8 is hydrolyzed with a basic to obtain the compound 9; and
(8) the compound 9 and aqueous solution containing calcium ions are salified to obtain the atorvastatin calcium.

Further, structures of the compounds 1 to 10 are as follows:

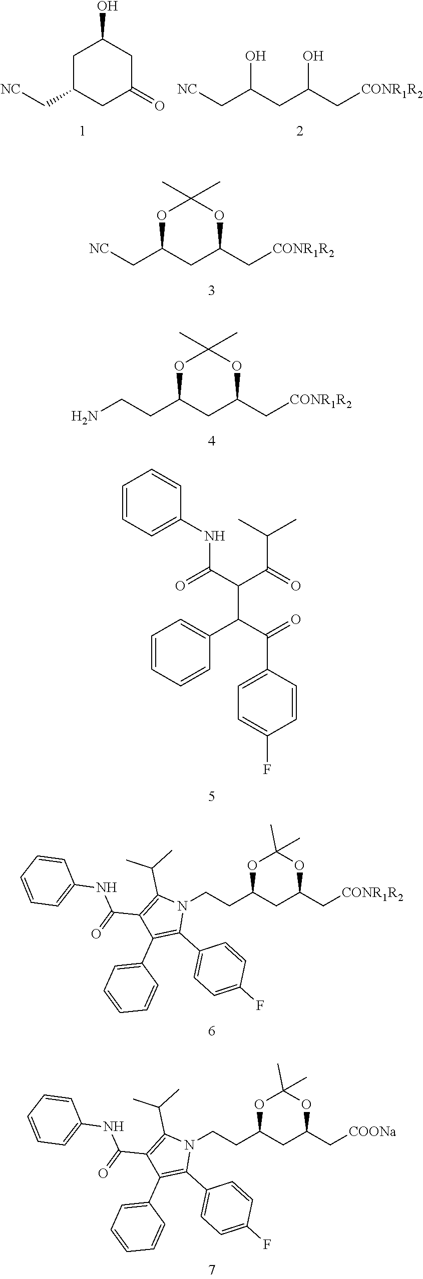

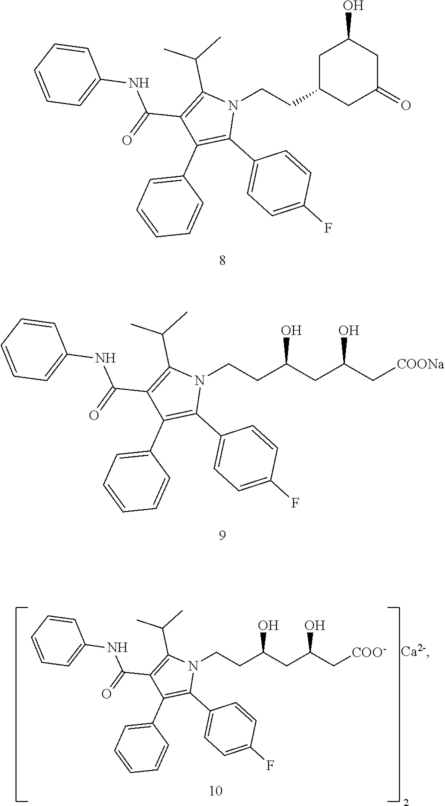

where, R1 and R2 are respectively C1~C6 alkyl.

Further, in Step (1), the ammonia compound is one of ammonia gas, C1~C6 primary amine and C1~C6 secondary amine.

Further, in Step (2), the acid is p-toluenesulfonic acid.

Further, in Step (3), the pressure is 0.1 MPa to 2 MPa.

Further, in Step (4), a temperature of the reaction is between 60° C. to 70° C.

Further, in Step (5), the alkali is one or more of sodium carbonate, potassium carbonate, sodium hydroxide, and potassium hydroxide.

Further, in Step (6), the acid is one or more of hydrochloric acid, sulfuric acid, sulfurous acid, and phosphoric acid.

Further, in Step (7), the alkali is one or more of sodium carbonate, potassium carbonate, sodium hydroxide, and potassium hydroxide.

Further, in Step (8), the solute of the aqueous solution containing calcium ions is one or more of calcium chloride, calcium acetate, calcium nitrate, and calcium gluconate.

Further, Step (5) and Step (6) are performed continuously in a same reactor.

Further, Step (7) and Step (8) are performed consecutively in a same reactor.

Further, in Step (6), a ratio of the compound 7 converted to the compound 8 is 10% to 20%, and in Step (7), the compound 8 is converted to the compound 9.

Compared with existing technologies, the beneficial effects of the present disclosure are as follows.
1. The advantage of using amide instead of tert-butyl ester in the process route of the present disclosure is that the compound 6 has a more stable structure and is easier to purify.
2. Avoiding the use of more expensive reagents, such as lithium diisopropylamide and sodium borohydride, the amide reaction conditions are mild, and the process is simple, making it suitable for industrial production.
3. In the process of converting the compound 6 to calcium salts, the yield is higher and the loss of raw material is lower.

### BRIEF DESCRIPTION OF THE DRAWINGS

Drawings described here are used to provide further understanding of the present application and constitute a part of the present application. Schematic embodiments of the present application and descriptions thereof are used to explain the present application and do not constitute improper limitations on the present application. In the drawings:

FIG. 1 is a nuclear magnetic resonance (NMR) of a compound 6 in Embodiment 1 of the present disclosure;

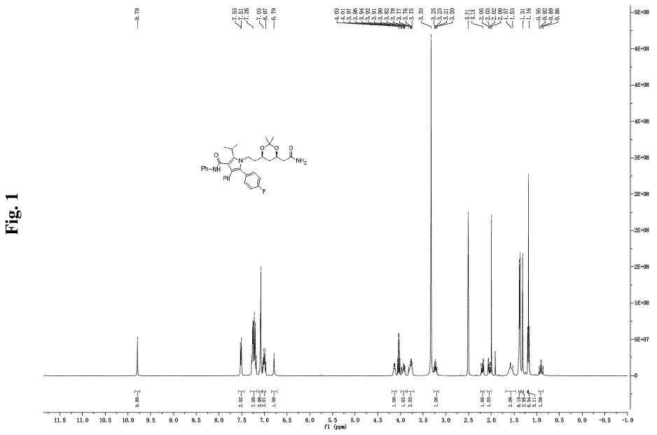

FIG. 2 is a nuclear magnetic resonance (NMR) of a compound 7 in Embodiment 1 of the present disclosure;

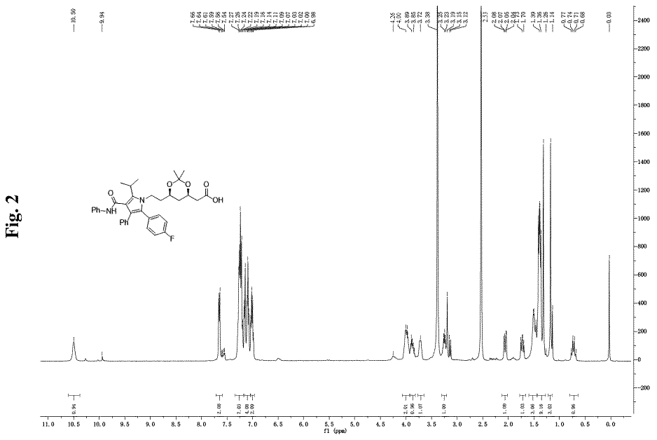

FIG. 3 is a nuclear magnetic resonance (NMR) of a compound 8 in Embodiment 1 of the present disclosure; and

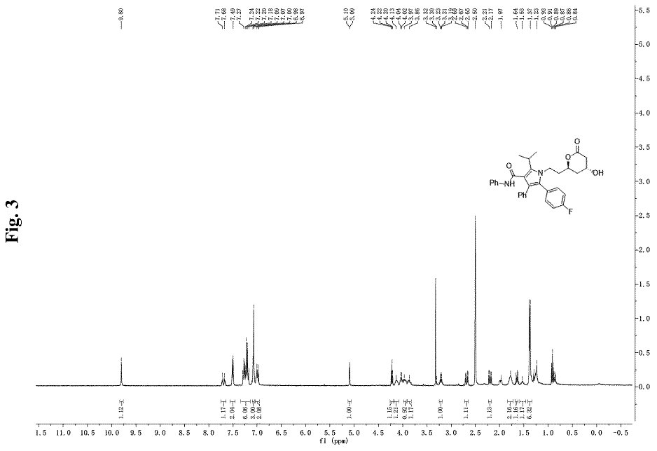

FIG. 4 is a nuclear magnetic resonance (NMR) of a compound 9 in Embodiment 1 of the present disclosure.

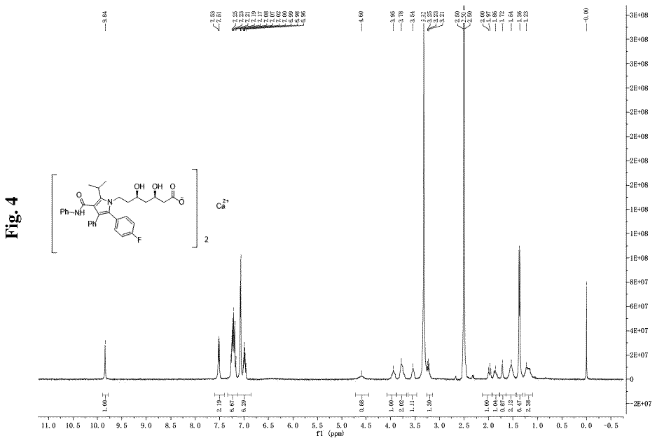

### DETAILED DESCRIPTION OF THE EMBODIMENTS

It should be noted that embodiments in the present application and features in the embodiments may be combined with each other in the case without conflicting. The present application is described in detail below with reference to the drawings and in combination with the embodiments.

It should be noted that terms used here are only intended to describe specific implementation modes and are not intended to limit the exemplary implementation modes according to the present application. As used herein, unless otherwise explicitly stated in the context, the singular form is also intended to include the plural form. Additionally, it should be understood that when the terms “containing” and/or “including” are used in the description, it indicates the presence of features, steps, operations, devices, components, and/or combinations thereof.

Unless otherwise specified, relative arrangements, numerical expressions, and values of the components and steps described in these embodiments do not limit the scope of the present application. At the same time, it should be understood that for ease of description, dimensions of various parts shown in the drawings are not drawn according to the actual proportional relation. For technologies, methods, and devices known to those of ordinary skill in the art, detailed discussions may not be made, but in the appropriate case, the technologies, methods, and devices should be considered as a part of the authorized description. In all examples shown and discussed here, any specific values should be explained as merely exemplary and not restrictive. Therefore, other examples of the exemplary embodiments may have different values. It should be noted that: similar labels and letters represent similar items in the following drawings. Therefore, once a certain item is defined in one drawing, it does not need to be further discussed in the subsequent drawings.

Technical schemes in the embodiments of the present disclosure are clearly and completely described below. Apparently, the embodiments described are only a part of the embodiments of the present disclosure, and not all of the embodiments. Based on the embodiments of the present disclosure, all other embodiments obtained by those of ordinary skill in the art without creative labor shall fall within the scope of protection of the present disclosure.

### Embodiment 1

A preparation method for an atorvastatin calcium intermediate included the following specific steps.

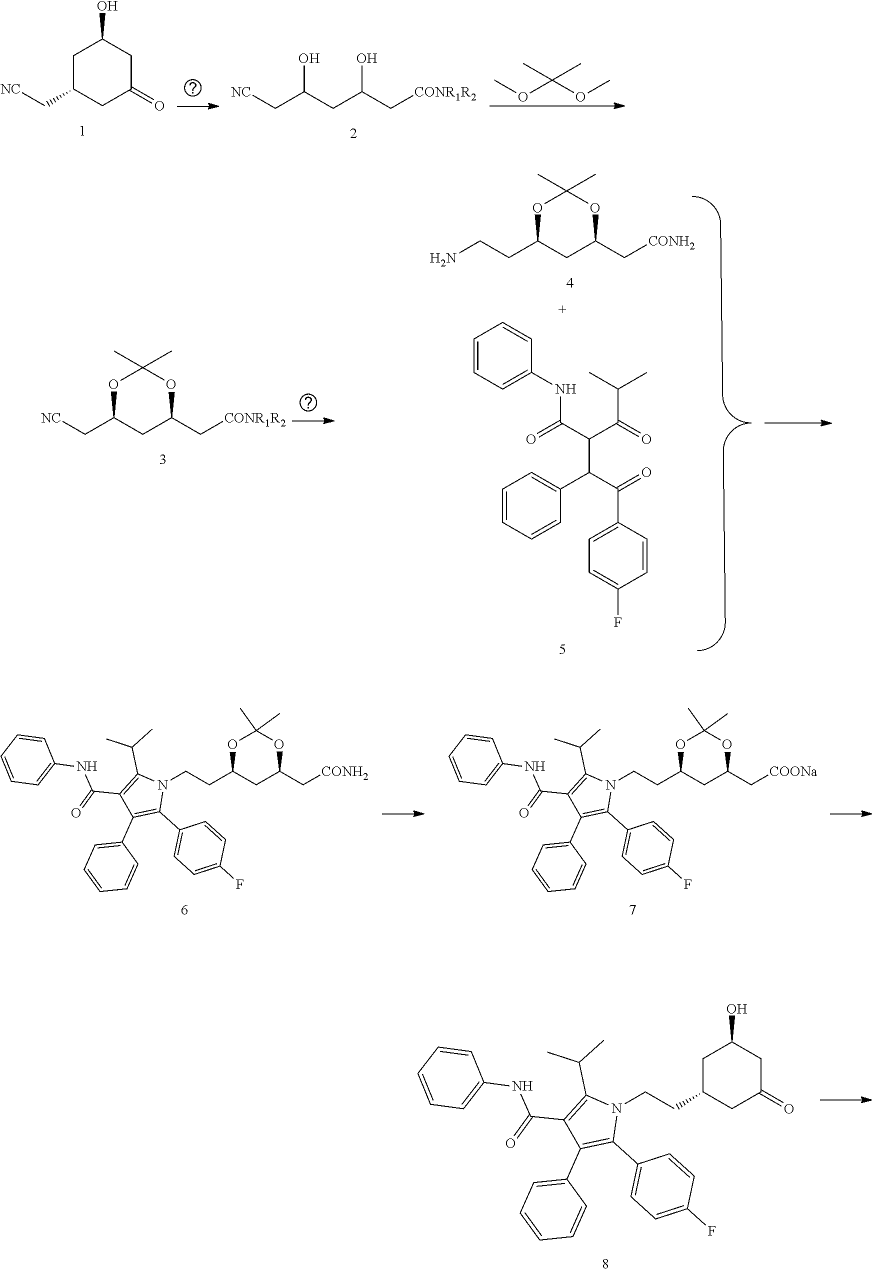

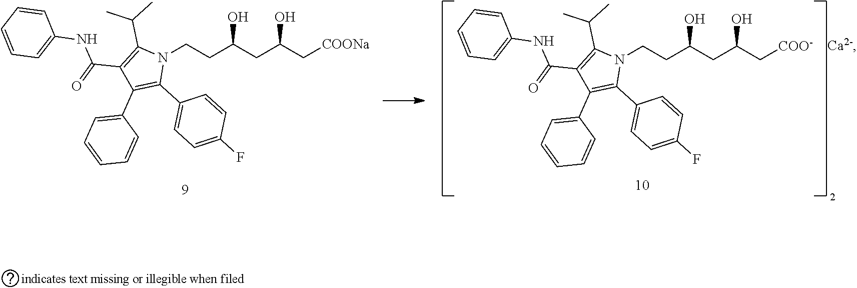

### (1) Synthesis of Compound 2

A compound 1 (50 g, 0.322 mol) and 10% ammonia ethanol (100 g, 0.588 mol) were added to a reaction flask, after being stirred for 10 min, the temperature was raised and a reflux reaction was performed for 5 h, it was reduced to a room temperature, and concentrated to dry, to obtain a compound 2 (55.19 g, 0.321 mol), the yield was 99.5% and the purity was 97.1%.

### (2) Synthesis of Compound 3

The compound 2 (50 g, 0.290 mol) and 200 mL dichloromethane were added to the reaction flask, after being stirred for 10 min, p-toluenesulfonic acid (0.5 g) was added, and after being stirred for 5 min, 2,2-dimethoxypropane (36.29 g, 0.348 mol) was added, it was reacted at the room temperature for 16 h, 50 mL of saturated sodium bicarbonate was added and stirred for 10 min, solution was separated, an aqueous layer was extracted once with dichloromethane, dichloromethane layers were combined, washed once with water, and spin-dried, to obtain a compound 3 (57.57 g, 0.271 mol), the yield was 93.4% and the purity was 97.5%.

### (3) Synthesis of Compound 4

The compound 3 (60 g, 0.283 mol), 12 g of Raney nickel, 20 g of 20% ammonia water, and 300 mL of methanol were added to a high-pressure reaction kettle, the air was replaced with a nitrogen gas, and the nitrogen gas was replaced with a hydrogen gas. The hydrogen gas was pressurized to 0.3 MPa, the temperature was raised to 50° C., and the process control pressure was stable. After being reacted for 5 h, it was reduced to the room temperature, and filtered, and a filtrate was spin-dried directly. Dichloromethane and water were added to separate layers, an organic layer was washed once with the water, and spin-dried, to obtain a compound 4 (59.43 g, 0.274 mol), the yield was 97.2% and the purity was 98.1%.

### (4) Synthesis of Compound 6

A compound 5 (50 g, 0.12 mol), the compound 4 (28.5 g, 0.13 mol), and 500 mL of cyclohexane were added to the reaction flask, after being stirred for 5 min, 25 mL of tetrahydrofuran was added, it was stirred continuously for 5 min, a trimethylacetic acid (6.12 g, 0.06 mol) was added, the temperature was raised and the reflux reaction was performed for 18 h. It was reduced to the room temperature, 250 mL of water was added, and it was stirred for 20 min. 10% sodium carbonate was added to adjust pH to 8.5 to 9.5, after layers were separated, an aqueous layer was extracted twice with dichloromethane, organic layers were combined, washed twice with water, the organic layer was spin-dried, after methanol was added for reflux and dissolved clarification, the temperature was reduced for crystallization, it was suction-filtered, and dried, to obtain a compound 6 (61.1 g, 0.102 mol), the yield was 85.3% and the purity was 99.4%.

### (5) Synthesis of Compound 8

The compound 6 (60 g, 0.1 mol), sodium hydroxide (4.42 g, 0.11 mol), and 300 mL of methanol were added to the reaction flask, it was heated and the reflux reaction was performed for 16 h, and it was reduced to the room temperature to obtain solution of a compound 7. The temperature was controlled and did not exceed 35° C., 25 mL of 2N hydrochloric acid solution was added, it was stirred and reacted at the room temperature for 4 h, and spin-dried, 300 mL of toluene and 1.2 g of p-toluenesulfonic acid were added, it was heated for reflux and water was separated for 8 h, it was reduced to the room temperature for crystallization, suction-filtered, and dried, to obtain a mixture of a compound 8 (5.4 g, 0.01 mol) and a compound 9 (46.5 g, 0.08 mol), the yield was 91.3%.

### (6) Synthesis of Compound 10

The mixture of the compound 8 (5.4 g, 0.01 mol) and the compound 9 (46.5 g, 0.08 mol) obtained in the previous step and 450 mL of methanol were added to the reaction flask, it was stirred at the room temperature for 10 min, and 46 mL of 2N sodium hydroxide aqueous solution was dropwise added. After being dropwise added, it was stirred at the room temperature for 2 h, methanol was spin-dried, 90 mL of water was added and 90 mL of methanol was added. After being stirred and dissolved, 40 mL of 2N calcium acetate aqueous solution was dropwise added slowly, and after being dropwise added, it was stirred for 2 h, suction-filtered, and dried, to obtain a compound 10 (48.9 g, 0.085 mol), the yield was 94.17% and the purity was 99.8%.

### Embodiment 2

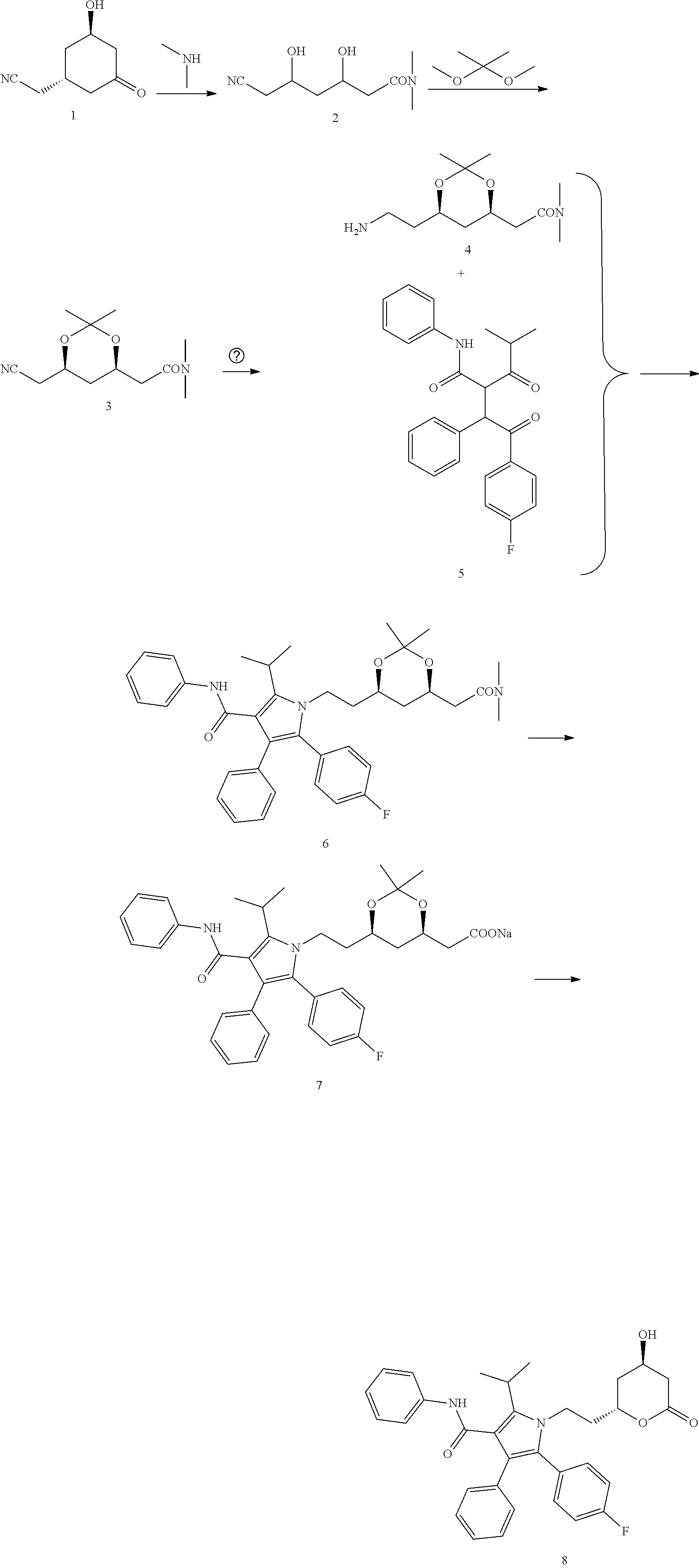

### (1) Synthesis of Compound 2

A compound 1 (50 g, 0.322 mol) and 10% dimethylamine ethanol solution (235 g, 0.588 mol) were, added to a reaction flask, after being stirred for 10 min, the temperature was raised and a reflux reaction was perform-ed for 5 h, it was reduced to a room temperature, and concentrated to dry, to obtain a compound 2 (63.83 g, 0.319 mol), the yield was 98.8% and the purity was 96.6%.

### (2) Synthesis of Compound 3

The compound 2 (58 g, 0.290 mol) and 200 mL dichloromethane were added to the reaction flask, after being stirred for 10 min, p-toluenesulfonic acid (0.5 g) was added, and after being stirred for 5 min, 2,2-dimethoxypropane (36.29 g, 0.348 mol) was added, it was reacted at the room temperature for 16 h, 50 mL of saturated sodium bicarbonate was added and stirred for 10 min, solution was separated, an aqueous layer was extracted once with dichloromethane, dichloromethane layers were combined, it was washed once with water, and spin-dried, to obtain a compound 3 (64.21 g, 0.267 mol), the yield was 92.1% and the purity was 97.2%.

### (3) Synthesis of Compound 4

The compound 3 (68 g, 0.283 mol), 12 g of Raney nickel, 20 g of 20% ammonia water, and 300 mL of methanol were added to a high-pressure reaction kettle, the air was replaced with a nitrogen gas, and the nitrogen gas was replaced with a hydrogen gas. The hydrogen gas was pressurized to 0.3 MPa, the temperature was raised to 50° C. and the process control pressure was stable. After being reacted for 5 h, it was reduced to the room temperature, and filtered, and a filtrate was spin-dried directly. Dichloromethane and water were added to separate layers, an organic layer was washed once with the water, and spin-dried, to obtain a compound 4 (66.19 g, 0.271 mol), the yield was 96.1% and the purity was 97.9%.

### (4) Synthesis of Compound 6

A compound 5 (50 g, 0.12 mol), the compound 4 (31.76 g, 0.13 mol), and 500 mL of cyclohexane were added to the reaction flask, after being stirred for 5 min, 25 mL of tetrahydrofuran was added, it was stirred continuously for 5 min, a trimethylacetic acid (6.12 g, 0.06 mol) was added, the temperature was raised and the reflux reaction was performed for 18 h. It was reduced to the room temperature, 250 mL of water was added, and it was stirred for 20 min. 10% sodium carbonate was added to adjust pH to 8.5 to 9.5, after layers were separated, an aqueous layer was extracted twice with dichloromethane, organic layers were combined, it was washed twice with water, the organic layer was spin-dried, after methanol was added for reflux and dissolved clarification, the temperature was reduced for crystallization, it was suction-filtered, and dried, to obtain a compound 6 (63.08 g, 0.101 mol), the yield was 84.3% and the purity was 99.2%.

### (5) Synthesis of Compound 8

The compound 6 (62.58 g, 0.1 mol), sodium hydroxide (4.42 g, 0.11 mol), and 300 mL of methanol were added to the reaction flask, it was heated and the reflux reaction was performed for 16 h, and it was reduced to the room temperature to obtain solution of a compound 7. The temperature was controlled and did not exceed 35° C., 25 mL of 2N hydrochloric acid solution was added, it was stirred and reacted at the room temperature for 4 h, and spin-dried, 300 mL of toluene and 1.2 g of p-toluenesulfonic acid were added, it was heated for reflux and water was separated for 8 h, it was reduced to the room temperature for crystallization, suction-filtered, and dried, to obtain a mixture of a compound 8 (5.4 g, 0.01 mol) and a compound 9 (45.9 g, 0.079 mol), the yield was 90.5%.

### Embodiment 3

Synthesis of Compound 4 (R1 and R2 were H)

A compound 3 (60 g, 0.283 mol), 12 g of Raney nickel, 20 g of 20% ammonia water, and 300 mL of methanol were added to a high-pressure reaction kettle, the air was replaced with a nitrogen gas, and the nitrogen gas was replaced with a hydrogen gas. The hydrogen gas was pressurized to 0.1 MPa, the temperature was raised to 50° C., and the process control pressure was stable. After being reacted for 8 h, it was reduced to the room temperature, and filtered, and a filtrate was spin-dried directly. Dichloromethane and water were added to separate layers, an organic layer was washed once with the water, and spin-dried, to obtain a compound 4 (59.26 g, 0.269 mol), the yield was 95.3% and the purity was 98%.

### Embodiment 4

Synthesis of Compound 4 (R1 and R2 were H)

A compound 3 (60 g, 0.283 mol), 12 g of Raney nickel, 20 g of 20% ammonia water, and 300 mL of methanol were added to a high-pressure reaction kettle, the air was replaced with a nitrogen gas, and the nitrogen gas was replaced with a hydrogen gas. The hydrogen gas was pressurized to 2 MPa, the temperature was raised to 50° C., and the process control pressure was stable. After being reacted for 5 h, it was reduced to the room temperature, and filtered, and a filtrate was spin-dried directly. Dichloromethane and water were added to separate layers, an organic layer was washed once with the water, and spin-dried, to obtain a compound 4 (60.58 g, 0.275 mol), the yield was 97.5% and the purity was 98.3%.

### Embodiment 5

Synthesis of Compound 6 (R1 and R2 were H)

A compound 5 (50 g, 0.12 mol), a compound 4 (28.5 g, 0.13 mol) and 500 mL of cyclohexane were added to a reaction flask, after being stirred for 5 min, 25 mL of tetrahydrofuran was added, it was stirred continuously for 5 min, a trimethylacetic acid (6.12 g, 0.06 mol) was added, the temperature was raised to 60° C. and it was reacted for 18 h. It was reduced to the room temperature, 250 mL of water was added, and it was stirred for 20 min. 10% sodium carbonate was added to adjust pH to 8.5 to 9.5, after layers were separated, an aqueous layer was extracted twice with dichloromethane, organic layers were combined, it was washed twice with water, the organic layer was spin-dried, after methanol was added for reflux and dissolved clarification, the temperature was reduced for crystallization, it was suction-filtered, and dried, to obtain a compound 6 (57.87 g, 0.097 mol), the yield was 80.8% and the purity was 98.4%.

The above are only preferred embodiments of the present application and are not used to limit the present application. For those skilled in the art, the present application may have various modifications and variations. Any modifications, equivalent replacements, improvements and the like made within the spirit and principles of the present application should be contained within the scope of protection of the present application.

1. A preparation method for atorvastatin calcium, the atorvastatin calcium is as follows: 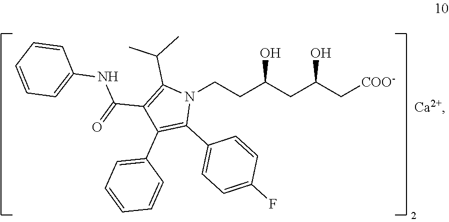
wherein comprising the following steps:
(1) a compound 1 is ring-cleaved by an ammonia compound to obtain a compound 2;
(2) the compound 2 is reacted with 2,2-dimethoxypropane in a catalytic reaction with an acid to obtain a compound 3;
(3) the compound 3 is catalytic-hydrogenated by Raney nickel under a pressure to obtain a compound 4;
(4) the compound 4 is condensed with a compound 5 to obtain a compound 6;
(5) the compound 6 is hydrolyzed to obtain a compound 7;
(6) the compound 7 is subjected to acid deprotection and acid catalytic dehydration to obtain a compound 8, while the compound 7 is subjected to the acid deprotection to obtain a compound 9;
(7) the compound 8 is hydrolyzed with a basic to obtain the compound 9; and
(8) the compound 9 and an aqueous solution containing calcium ions are salified to obtain the atorvastatin calcium.

2. The preparation method for the atorvastatin calcium according to claim 1, wherein structures of the compounds 1 to 10 are as follows: 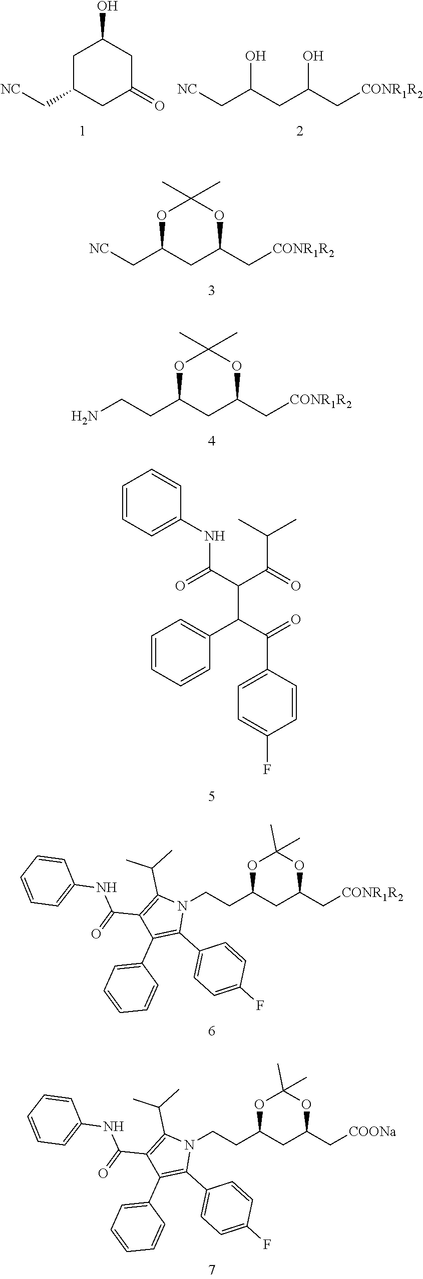 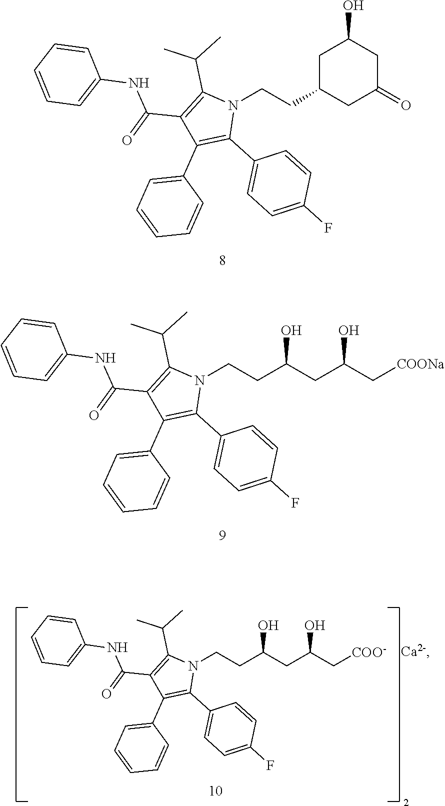
wherein, R1 and R2 are respectively C1~C6 alkyl.

3. The preparation method for the atorvastatin calcium according to claim 1, wherein in Step (1), the ammonia compound is one of ammonia gas, C1~C6 primary amine and C1~C6 secondary amine.

4. The preparation method for the atorvastatin calcium according to claim 1, wherein in Step (2), the acid is p-toluenesulfonic acid.

5. The preparation method for the atorvastatin calcium according to claim 1, wherein in Step (3), the pressure is 0.1 MPa to 2 MPa.

6. The preparation method for the atorvastatin calcium according to claim 1, wherein in Step (4), a temperature of reaction is between 60° C. to 70° C.

7. The preparation method for the atorvastatin calcium according to claim 1, wherein in Step (5), the compound 6 is hydrolyzed with an alkali to obtain the compound 7, the alkali is one or more of sodium carbonate, potassium carbonate, sodium hydroxide, and potassium hydroxide.

8. The preparation method for the atorvastatin calcium according to claim 1, wherein in Step (6), an acid of the acid deprotection is one or more of hydrochloric acid, sulfuric acid, sulfurous acid, and phosphoric acid.

9. The preparation method for the atorvastatin calcium according to claim 1, wherein in Step (7), the alkali is one or more of sodium carbonate, potassium carbonate, sodium hydroxide, and potassium hydroxide.

10. The preparation method for the atorvastatin calcium according to claim 1, wherein in Step (8), a solute of the aqueous solution containing calcium ions is one or more of calcium chloride, calcium acetate, calcium nitrate, and calcium gluconate.

11. The preparation method for the atorvastatin calcium according to claim 1, wherein Step (5) and Step (6) are performed continuously in a same reactor.

12. The preparation method for the atorvastatin calcium according to claim 1, wherein Step (7) and Step (8) are performed consecutively in a same reactor.

13. The preparation method for the atorvastatin calcium according to claim 1, wherein in Step (6), a ratio of the compound 7 converted to the compound 8 is 10% to 20%, and in Step (7), the compound 8 is converted to the compound 9.

14. The preparation method for the atorvastatin calcium according to claim 2, wherein in Step (3), the pressure is 0.1 MPa to 2 MPa.

15. The preparation method for the atorvastatin calcium according to claim 3, wherein in Step (3), the pressure is 0.1 M Pa to 2 MPa.

16. The preparation method for the atorvastatin calcium according to claim 2, wherein in Step (4), a temperature of reaction is between 60° C. to 70° C.

17. The preparation method for the atorvastatin calcium according to claim 3, wherein in Step (4), a temperature of reaction is between 60° C. to 70° C.

18. The preparation method for the atorvastatin calcium according to claim 2, wherein in Step (5), the compound 6 is hydrolyzed with an alkali to obtain the compound 7, the alkali is one or more of sodium carbonate, potassium carbonate, sodium hydroxide, and potassium hydroxide.

19. The preparation method for the atorvastatin calcium according to claim 3, wherein in Step (5), the compound 6 is hydrolyzed with an alkali to obtain the compound 7, the alkali is one or more of sodium carbonate, potassium carbonate, sodium hydroxide, and potassium hydroxide.

20. The preparation method for the atorvastatin calcium according to claim 2, wherein in Step (6), the acid is one or more of hydrochloric acid, sulfuric acid, sulfurous acid, and phosphoric acid.
